(19) United States
(12) Patent Application Publication
(10) Pub. No.: US 2005/0241637 A1
(43) Pub. Date: Nov. 3, 2005

(54) Metered dose inhaler

(76) Inventor: Wendy W. Westrate, Aiken, SC (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/106,226

(22) Filed: Apr. 14, 2005

Related U.S. Application Data
(60) Provisional application No. 60/567,414, filed on Apr. 29, 2004.

(51) Int. Cl. A61M 11/00

(52) U.S. Cl. 128/200.23

(57) ABSTRACT

A metered dose inhaler including a housing having a mouth piece end and a receiving end is provided. The mouth piece end includes a mouth piece and a cover for the mouth piece. The receiving end is adapted to receive a canister. The metered dose inhaler includes an attachment, which attaches to the receiving end of the metered dose inhaler. The attachment provides an increased gripping surface and generally provides a non-slip surface to aid in the proper delivery of a metered dose of the substance to be delivered.

### CROSS-REFERENCE TO RELATED APPLICATION

This application claims the benefit of U.S. Provisional Patent Application No. 60/567,414, filed Apr. 30, 2004, which is hereby incorporated by reference in its entirety.

### FIELD OF THE INVENTION

The present invention is directed toward metered dose inhalers having an attachment connected thereto and to attachments for metered-dose inhalers (hereinafter MDI).

### BACKGROUND OF THE INVENTION

MDIs are devices that help deliver a specific amount of medication in aerosol form to the lungs. This makes it possible to inhale the medication, instead of taking it in pill form. MDIs are commonly used to treat asthma, chronic obstructive pulmonary disease and other respiratory problems. A MDI typically includes five parts: the medication, the propellant, the canister, the metering device and the mouthpiece. Various attachments for MDIs are known. One such attachment is a spacer which is attached to the mouth piece of an MDI. Typically, when an MDI is used without a spacer, because the medication is stored at such high pressures within the MDI canister, when the MDI is activated and the medication is released from the canister a large portion of the metered dose released from the canister is impacted on the back of a patient's throat. This is in part due to the high rate of speed at which the metered dose is released from the canister. Therefore, spacer attachments are designed to increase the distance the medication has to travel so the medication slows down (i.e., decreases in speed) before it enters the patient's mouth. This decreases impaction on the back of a patient's throat and delivers more of the medication to the intended delivery site (generally the lungs of a patient).

However, there are no known attachments that fit the canister end of an MDI housing. Surprisingly, Applicant has discovered a novel attachment device, which fits on the canister end of an MDI, which increases the gripping surface and generally provides a non-slip surface for patients when using an MDI.

### SUMMARY OF THE INVENTION

One aspect of the present invention includes an MDI having a housing. The housing includes a mouth piece end and a receiving end. The mouth piece end of the housing further includes a mouth piece cover for the mouth piece. The receiving end is adapted to receive a canister. The MDI also includes an attachment, which attaches to the receiving end of the MDI and provides an increased gripping surface, and generally provides a non-slip surface to aid in the proper delivery of the substance to be delivered.

Another aspect of the present invention includes a MDI attachment adapted to attach to the receiving end of a MDI, which provides an increased gripping surface, and generally provides a non-slip surface, to ensure proper delivery of the metered dose of the substance to be delivered.

Yet another aspect of the present invention includes a method of properly administering a metered dose to a patient providing a MDI having a housing. The housing includes a mouth piece end and a receiving end. The mouth piece end further includes a mouth piece cover for the mouth piece. The receiving end is adapted to receive a canister. The MDI also includes an attachment, which attaches to the receiving end of the MDI and provides an increased gripping surface, and generally provides a non-slip surface to aid in the proper delivery of the medication to be delivered. A patient then depresses the attachment and canister of the MDI to discharge a metered dose.

These and other features, advantages, and objects of the present invention will be further understood and appreciated by those skilled in the art by reference to the following specification, claims, and appended drawings.

### BRIEF DESCRIPTION OF THE DRAWINGS

FIG. 1 is an exploded side view of the MDI including an attachment on the receiving end of the housing according to one aspect of the present invention;

FIG. 2 is a perspective view of the MDI attachment device according to one aspect of the present invention;

FIG. 3 is a perspective view of the MDI including an attachment device attached thereto according to one aspect of the present invention;

FIG. 4 is a view of the MDI attachment device taken along the line IV-IV; FIG. 2;

FIG. 5 is an end view of an MDI attachment device according to another aspect of the invention.

### DETAILED DESCRIPTION OF PREFERRED EMBODIMENT

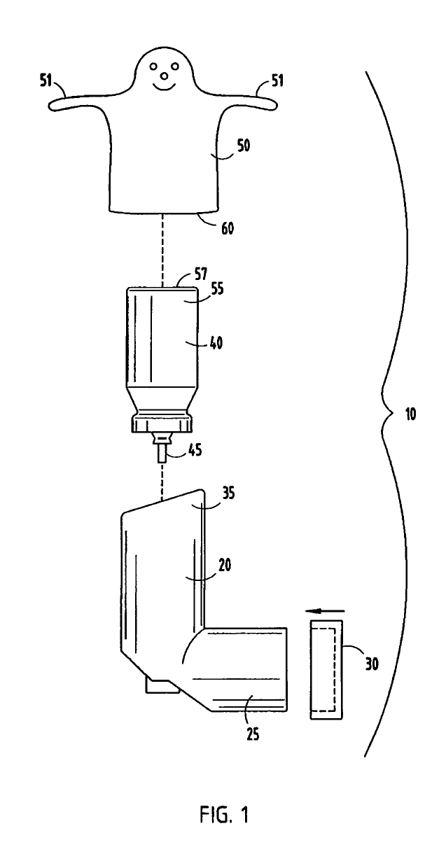

For purposes of description herein, the terms “upper,” “lower,” “right,” “left,” “rear,” “front,” “vertical,” “horizontal,” and derivatives thereof shall relate to the invention as oriented in FIG. 1. However, it is to be understood that the invention may assume various alternative orientations, except where expressly specified to the contrary. It is also to be understood that the specific devices and processes illustrated in the attached drawings and described in the following specification are exemplary embodiments of the inventive concepts defined in the appended claims. Hence, specific dimensions and other physical characteristics relating to the embodiments disclosed herein are not to be considered as limiting, unless the claims expressly state otherwise.

Referring to FIG. 1, reference numeral 10 designates an MDI according to one aspect of one embodiment of the present invention. MDI 10 includes a mouthpiece or housing 20, cover 30, canister 40 and an attachment device 50.

The overall shape of housing 20 of MDI 10 may be any shape, including, but not limited to, rectangular, oblong, oval, L-shaped, etc., or any combinations or derivations of any of these shapes. However, L-shaped is preferred. Housing 20 may be made of any materials, including, but not limited to, plastic, metal, wood, etc. or any combinations or derivations thereof, however, plastic is preferred.

Housing 20 of MDI 10 includes a mouth piece end 25 and a receiving end 35 (see FIG. 1). Mouth piece end 25 may be any shape, including, but not limited to, square, rectangular, circular, oval, trapezoidal, etc., however, mouth piece end 25 is preferably oval in shape. The shape of mouth piece end 25 may be any shape and size so long as the patient can put their mouth around mouth piece end 25. Removably attached to mouth piece end 25 may be cover 30. Cover 30 may be any shape so long as it may be receivably attached to mouth piece end 25. The shape of receiving end 35 may be any shape so long as receiving end 35 may receive canister 40. Receiving end 35 may be square, rectangular, circular, oval, trapezoidal, etc., however, circular-shaped is preferred.

Canister 40 includes a first end 45 and a second end 55. First end 45 includes an outlet for releasing the contents of canister 40. That is to say, upon exerting pressure in a generally downward motion such that first end 45 of canister 40 contacts at least a portion of housing 20 with an amount of pressure required to release the substance from within canister 40. This is generally known in the art. The surface 57 of second end 55 is generally flat, concave or convex. Canister 40 may include any known medication suitable for metered dose inhalation. Canister 40 may be made from any material, including, but not limited to, metal, wood, plastic, etc., or any combinations or derivations thereof, however, plastic is preferred. Canister 40 may be any shape, including, but not limited to, cylinder shaped, rectangular shaped, etc., however, cylindrically shaped is preferred.

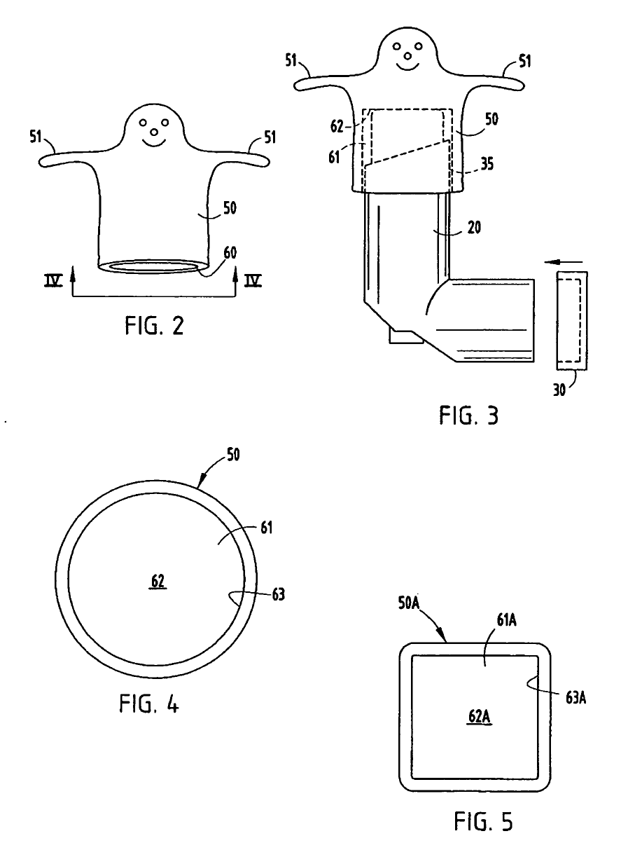

Attachment 50 includes at least one open end 60 (see FIGS. 1 and 2). Open end 60 is sufficiently large enough to fit over second end 55 of canister 40. Alternatively, open end 60 may be large enough in size to fit over both second end 55 of canister 40 and over receiving end 35 of housing 20. The “fit” referred to herein regarding attachment device 50 generally includes any type of connection to either/both second end 55 of canister 40 and receiving end 35 of housing 20. Such a fit may include, but is not limited to, a tension fit, an adhesion fit, a suction fit, etc., or any combinations or derivations herein, however, a tension fit is preferred. Open end 60 opens to an internal cavity formed in attachment device 50. An end surface 62 of cavity 61 abuts surface 57 of canister 40, such that a downward force on device 50 causes canister 40 to be shifted downwardly, thereby releasing a metered dose of medication.

With further reference to FIG. 4, cavity 61 may be defined by a cylindrical internal surface 63 that fits closely, yet slidably around a housing 20 having a cylindrically-shaped receiving end 35. Alternately, an attachment device 50A has inner surfaces 63A defining a cavity 62A having a quadrilateral shape that fits closely, yet slidably, around a receiving end of a housing 20 having a quadrilateral cross-sectional shape. Attachment device 50 typically is made of material is easily gripped and that generally provides a non-slip surface. Such materials, include, but are not limited to, silicone, rubber, plastic, wood, metal, etc., or any combinations or derivations herein, however, silicone or rubber are preferred. Device 50 may be malleable or semi-malleable. Device 50 may be any shape, including, but not limited to, characters (i.e., cartoon, comic, movie, etc.), animals, toys, or any geometric shape, etc., or any derivations or combinations of any of these, however, animal shapes are preferred. Device 50 may also include a variety of non-slip exterior textures or exterior texture enhancers, including, but not limited to, surface bubbles, partial surface bubbles, adhesives, etc., however, partial surface bubbles are preferred. Also, device 50 may include one or more protrusions 51 or the like that form grips or handles to further facilitate activation of the MDI.

Another aspect of the present invention includes a method of properly administering a metered dose of a substance to be delivered to a patient. Such a method includes the MDI of the present invention. The MDI of the present invention includes an attachment, which attaches to the receiving end of the MDI and provides an increased gripping surface, and generally provides a non-slip surface, to aid in the proper delivery of the metered dose of the medication to be delivered. A patient then grips the device and depresses the canister of the MDI to discharge a metered dose.

In the foregoing description, it will be readily appreciated by those skilled in the art that modifications may be made to the invention without departing from the concepts disclosed herein. Such modifications are to be considered as included in the following claims, unless these claims by their language expressly state otherwise.

1. A metered dose inhaler, comprising:
a housing including a mouth piece end and a receiving end, wherein the mouth piece end includes a cover in communication with the mouth piece end and wherein the receiving end is adapted to receive a canister;
a canister having pressurized medicants therein, and having a first end and a second end having a valve configured to selectively release the medicant when activated to provide a metered dose of medicant in aerosol form from the mouth piece end of the housing, wherein a portion of the canister is received in the receiving end of the housing; and
an attachment member movably associated with the receiving end of the housing, wherein the attachment member includes an engagement surface abutting the canister, such that movement of the attachment member shifts the canister and activates the valve.

2. The metered dose inhaler of claim 1, wherein:
the housing comprises an L-shape.

3. The metered dose inhaler of claim 2, wherein:
the attachment member is slidably interconnected with the receiving end of the housing.

4. The metered dose inhaler of claim 1, wherein:
the canister defines an end surface; and
the engagement surface contacts the end surface.

5. The metered dose inhaler of claim 1, wherein:
the attachment member defines a cavity; and
a portion of the canister is received in the cavity.

6. The metered dose inhaler of claim 5, wherein:
the receiving end of the housing is disposed in the cavity.

7. The metered dose inhaler of claim 6, wherein:
the cavity has a cylindrical sidewall surface.

8. The metered dose inhaler of claim 1, wherein:
the attachment member includes a body portion and a pair of extensions protruding from the body portion to provide an increased gripping area.

9. The metered dose inhaler of claim 1, wherein:
the attachment member has a shape forming a figurine.

10. In a metered dose inhaler of the type having an L-shaped housing defining an upwardly extending canister-receiving end and a laterally extending open end, and a canister having pressurized medicants therein, the canister engaging the canister-receiving end of the housing, the improvement comprising:
an attachment member movably associated with the canister-receiving end of the metered dose inhaler housing, wherein the attachment member pushes against an upper end of the canister to release a metered dose of the medicant, and wherein the attachment member provides an increased gripping surface relative to a gripping surface of the canister.

11. The metered dose inhaler of claim 10, wherein:
the attachment member defines a cavity; and
a portion of the canister is received in the cavity.

12. The metered dose inhaler of claim 11, wherein:
the canister defines an end surface; and
the cavity of the attachment member defines an inner surface that abuts the end surface of the canister.

13. The metered dose inhaler of claim 12, wherein:
the canister-receiving end of the housing has a cylindrical outer surface; and
the cavity defines a cylindrical wall surface portion fitting closely around the cylindrical outer surface.

14. The metered dose inhaler of claim 10, wherein:
the attachment member includes a body portion and a pair of extensions that extend outwardly from the body portion.

15. The metered dose inhaler of claim 10, wherein:
the attachment member is shaped to form a figurine.

16. A method of administering a metered dose of medicant from a metered dose inhaler, comprising:
providing a metered dose inhaler including a housing having a mouth end and a receiving end adapted to receive a canister;
providing an attachment member having an increased gripping surface;
attaching the attachment member to the metered dose inhaler adjacent to the receiving end of the housing; and
depressing the attachment member and canister to discharge a metered dose.

17. The method of claim 16, wherein:
the attachment member has an opening; and
at least a portion of the canister is received in the opening when the attachment member is attached to the metered dose inhaler.

18. The method of claim 17, wherein:
at least a portion of the receiving end of the housing is received in the opening.

19. The method of claim 16, wherein:
the attachment member is attached to the metered dose inhaler by sliding the attachment member over the receiving end of the housing.

20. The method of claim 16, wherein:
the attachment member includes a pair of extensions; and
the attachment member is depressed by pushing on the extensions.
